(19) United States
(12) Patent Application Publication
(10) Pub. No.: US 2009/0171305 A1
(43) Pub. Date: Jul. 2, 2009

(54) COMBINATION THERAPY FOR LONG-LASTING CKR

(76) Inventor: Sami G. El Hage, Houston, TX (US)

Correspondence Address: FAEGRE & BENSON LLP;PATENT DOCKETING - INTELLECTUAL PROPERTY, 2200 WELLS FARGO CENTER, 90 SOUTH SEVENTH STREET, MINNEAPOLIS, MN, 55402-3901, US

(21) Appl. No.: 12/159,860

(22) PCT Filed: Jan. 4, 2007
(86) PCT No.: PCT/US07/60082

§ 371 Date: Jan. 23, 2009

Related U.S. Application Data
(60) Provisional application No. 60/756,334, filed on Jan. 5, 2006.
(60) Provisional application No. 60/874,040, filed on Dec. 9, 2006.

(51) Int. Cl. A61F 9/013; A61F 9/00

(52) U.S. Cl. 604/294; 604/22; 604/20

(57) ABSTRACT

Corneal collagen crosslinking of CKR™-treated cornea results in a long-lasting, potentially permanent correction of corneal shape and improved vision.

### BACKGROUND OF THE INVENTION

Visual or optical defects that prevent parallel light rays entering the eye from focusing clearly on the retina exist in several varieties. In hyperopia (farsightedness), the point of focus lies behind the retina, generally because the axis of the eyeball is too short. In myopia (nearsightedness), the image is focused in front of the retina, generally because the axis of the eyeball is too long. In astigmatism, refraction is unequal on the different meridians of the eyeball, generally due to asymmetry in the shape of the eye.

Corrective glasses or contact lenses have been used to correct these defects, including convex (plus) lenses for hyperopia, concave (minus) lenses in myopia, and cylindrical lenses in astigmatism.

Surgical techniques such as myopic or hyperopic keratomileusis have been used to alter cornea curvature and thereby improve refractive error. The keratomileusis method cuts and removes a predicted thickness of the corneal disk with a microkeratome. Additional surgical procedures such as radial keratotomy use microincisions in the cornea to surgically modify the curvature of the cornea and thereby reduce or eliminate myopia or astigmatism. Other surgical techniques include photorefractive keratectomy (PRK) which uses a laser to ablate the center of the cornea and thus change the cornea. In Automated Lamilar Keratectomy (ALK) pressure is placed on the cornea to bulge the central dome. A flap in the dome is then opened, layers of corneal tissue are removed and the flap is then closed. Procedures combining aspects of ALK/PRK are sometimes used, called LASIK (laser in situ keratectomy).

Non-surgical techniques to improve refractive errors of the eye include orthokeratology. A specialized non-surgical method, termed “controlled kerato-reformation” (CKR™) is a procedure based on analysis of a patient's total corneal topography and shape factor (SF) instead of keratometry. CKR™ uses computer-assisted video-keratography and software (CornealMap) to produce a comprehensive topographical map of the patient's cornea, permitting more accurate design, fitting, and monitoring of corneal changes due to CKR™ contact lenses over time. The reshaping of the cornea and improved visual acuity achieved by wearing of the CRK lens(es) for a period of time is maintained on removal of the lens(es) for a period of time.

U.S. Pat. No. 5,695,509 discloses that a patient may achieve a desired CKR™ induced cornea reshaping by wearing reverse-geometry lenses for a period of time, e.g., approximately three to eight hours per day, with improved vision maintained during periods when not wearing the lens. In some instances a patient may wear the reverse-geometry lenses one or two nights a week or every night during sleep to maintain the desired shape of the cornea and functional vision.

It would be of great utility to provide a more permanent non-surgical method for reshaping the cornea and thereby effecting correction of visual defects.

### SUMMARY OF THE INVENTION

The methods of the present invention provide a more long-lasting and potentially permanent non-surgical reshaping and alteration of the curvature of the cornea. It has now been found that CKR™ treatment in combination with riboflavin and UV light is effective to extend the time of visual correction, possibly permanently. As embodied and broadly described herein, one aspect of the invention is directed to methods for extending the period of time a corrected shape of a cornea and/or improved vision is maintained following wear and removal of a CKR™ lens, for example, a reverse-geometry lens.

One method of the invention comprises fitting the patient with reverse geometry contact lens(es) designed and fitted according to controlled kerato-reformation (CKR™) methods (or any reverse geometry contact lens). CKR™ is an orthokeratology procedure based on computer-assisted video-keratography including total corneal topography and shape-factor. The corneal reshaping is monitored for a period of time until a desired reshaping and/or visual improvement is reached. When the patient's cornea(s) has achieved a desired reshaping and/or a desired improvement in vision has been achieved, a cross-linking agent such as riboflavin and UV light is applied to the reshaped cornea to induce greater rigidity of the cornea tissue. As show in the Examples below, such cross-linking results in a longer-lasting and potentially permanent maintenance of corneal reshaping and/or visual improvement.

In one embodiment, a photosensitizer solution comprising dextran and riboflavin, for example, 0.1% riboflavin (10 mg riboflavin-5-phosphate) and 10 mL 20% dextran-T-500, is administered to the eye for a time sufficient for the riboflavin to reach the anterior chamber of the eye, for example 10-15 minutes or more. A slit-lamp can be used to confirm the presence of the cross-linking agent in the anterior chamber of the eye. The riboflavin treated cornea with is exposed to UV light, for example, using an LED radiating light at about 360 to about 370 nanometers, preferably about 365 nm, with an intensity of about 3 mW/cm². The UV exposure is generally for at least about 30 minutes, but can be more or less, as needed.

In an embodiment, the corneal epithelium is debrided or cut to enhance penetration of the crosslinking agent.

In one embodiment, the cross-linking agent is applied in an amount sufficient to saturate the cornea and/or be present in the anterior chamber of the eye. When the crosslinking agent is Riboflavin/UV, the riboflavin is applied in an amount and manner that provides a yellow coloration in the auterior chamber of the cornea when viewed, for example, via a slit-lamp. Preferably, the corneal tissue and the anterior chamber show a similar or the same yellow coloration due to riboflavin. In another embodiment, the cross-linking agent is applied in an amount and manner sufficient to increase the rigidity of the cornea (R). The crosslinking agent preferably penetrates at least ¾ of the corneal tissue, for example, at least 300 mm.

In one embodiment, the desired shape and/or improvement in vision is maintained for at least 1 month, 6 months, or one year or permanent. In an embodiment, the crosslinking of a CKR™-reshaped cornea reduces the periodicity of CKR™ lens wear needed to maintain the reshaping and/or corrected vision from about once daily to about once per week or about once per month, or even longer.

In one embodiment, the patient's corneal topography is analyzed using an EH-300 corneal topography map analyzer.

In one embodiment, the combination method provides visual improvement for myopia, astigmatism, or both.

### BRIEF DESCRIPTION OF THE DRAWINGS

FIG. 1 is a diagram showing the structure of a reverse-geometry lens.

FIG. 2 is a photograph of a computer image showing centration of a 4-zone OK lens on a patient's eye.

FIG. 3 is a photograph showing an apparatus for measuring corneal topography.

FIG. 4 is a photograph of a computer screen showing results from the CKR™ software program NOMOGRAM, displaying target myopia reduction, overall diameter of the contact lens (OAD), back optical zone radius (BOZR), location of anchorage zone (AC), width and height of invagination (RC), back optical zone diameter (BOZD), peripheral curve (PC), and slope. Also shown is the fluorescein pattern of the contact lens on the cornea, and below this, the tear layer thickness (TLT). Values of the shape factor (SF), eccentricity, and Q are also shown at the bottom of the display.

FIG. 5 is a photograph of a computer screen showing corneal topography of a well-centered orthoK-K CKR™ contact lens, with nice central flattening of the cornea, followed by a steeper zone (the knee) and again a flat zone to the periphery.

FIG. 6 is a photograph of a computer screen showing a “smiley face” pattern of corneal topography from a cornea where the contact lens became decentralized during sleep. An upper decentration of the contact lens (an excessive flattening) is shown.

FIG. 7 is a photograph of a computer screen showing a “frowney face” pattern of corneal topography from a cornea where the contact lens became decentralized during sleep. A downward decentration of the contact lens (a steepening of the lens) is shown.

FIG. 8 is a photograph of a computer screen showing a “central island” pattern of corneal topography from a cornea, similar to that seen in post surgical keratorefractive procedures.

FIG. 9 is a diagram showing axes of measurement of the eye.

FIG. 10 is a photograph showing an Optivision CornealMap CKR™ Lens fitting nomogram.

FIG. 11 is a graph showing increase in uncorrected visual acuity for 20 subjects wearing CKR™™ lenses for overnight orthokeratology over a 6-month period.

FIG. 12 is a graph showing reduction in myopia for 20 subjects wearing CKR™™ lenses for overnight orthokeratology over a 6-month period.

FIG. 13 is a graph showing change in refractive error as a function of original refractive error for the refractive error data shown in FIG. 12.

FIG. 14 is a graph showing changes in corneal topography shape factor (1-e²) for 20 subjects wearing CKR™™ lenses for overnight orthokeratology over a 6-month period.

FIG. 15 is a photograph showing changes in wavefront aberrations pre and post CKR™™ treatment.

FIG. 16 is a graph showing Zernike coefficients pre and post CKR™™ treatment.

FIG. 17 is a graph showing central total corneal thickness pre and post CKR™™ treatment.

FIG. 18 is a graph showing peripheral (Inferior) total corneal thickness pre and post CKR™™ treatment.

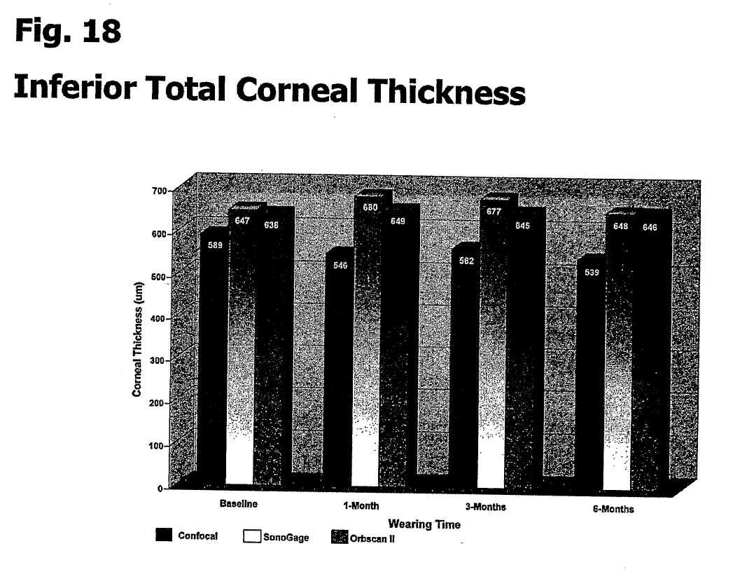

FIG. 19 is a graph showing central corneal epithelial thickness pre and post CKR™™ treatment.

FIG. 20 is a graph showing peripheral (inferior) corneal epithelial thickness pre and post CKR™™ treatment.

FIG. 21 is a graph showing the subjective comfort rating of the CKR™™ Lenses over time (0=no tolerable, 10=unable to feel).

FIG. 22 is a photograph of a corneal topography map showing a Difference Plot of a Patient A's cornea pre-CKR™ treatment and post-CKR™ treatment, with vector analysis.

FIG. 23 is a photograph of a corneal topography map showing Side by Side Plots of a Patient B's cornea pre-CKR™ treatment and post-CKR™ treatment.

FIG. 24 is a photograph of a corneal topography map showing Side by Side Plots of a Patient B's cornea pre- and post-crosslinking treatment.

FIG. 25 is a photograph of a corneal topography map showing a Summary Asymmetrical Refractive Plot of a Patient B's cornea pre-CKR™ treatment.

FIG. 26 is a photograph of a corneal topography map showing Fourier Analysis a Patient B's cornea pre-CKR™ treatment.

FIG. 27 is a photograph of a corneal topography map showing a Summary Symmetrical Refractive Plot of a Patient B's cornea 12 days post crosslinking treatment.

FIG. 28 is a photograph of a corneal topography map showing Fourier Analysis a Patient B's cornea at 12 days post-crosslinking treatment.

FIG. 29 is a photograph of a corneal topography map showing Side by Side Dioptric Plots of a Patient C's cornea at day one and at day 12 and 3 months post-crosslinking treatment.

FIG. 30 is a photograph of a corneal topography map showing Difference Plots of a Patient C's cornea at day one and at day 12 and 3 months post-crosslinking treatment.

FIG. 31 is a photograph of a corneal topography map showing Reversal of Asymmetrical Astigmatism of Patient C's cornea from the pre-CKR™ topography to the topography present at 10 days post-crosslinking treatment.

### DETAILED DESCRIPTION OF THE INVENTION

### Definitions

“Controlled kerato-reformation” (CKR™) is a non-invasive, non-surgical, reversible keratorefractive procedure for reducing myopia and improving vision. See, for example, U.S. Pat. No. 5,695,509.

“Crosslinking”, as used herein, is meant to define a chemical procedure that affects the rigidity of the cornea.

“Periodicity”, as used herein, is the frequency of lens wear versus non-lens wear necessary to maintain corneal reshaping and visual improvement. For example, once a desired corneal reshaping has been obtained via CKR™™ methods, a patient may wear the CKR™™ lenses about once daily (e.g., overnight) to maintain the desired reshaping and corrected vision during daily non-wear. After crosslinking of the cornea, this periodicity is reduced, for example, one overnight per week or per month, or even less frequently.

“R Factor”, as used herein, is a numerical calculation of the rigidity of the cornea, and is determined as a function of rigidity, flexibility, historesis, and reverse elasticity. R factor is determined by measuring the difference between the first and second applanation in function of time in a non-contact tonometer.

“Shape Factor” SF(ρ), as used herein, is a numerical calculation of the asphericity (Q) of the cornea, where the SF(ρ) is related to eccentricity (e).

ρ=1−e² and Q=−e²

### Controlled Kerato-Reformation (CKR™) and Cross-Linking

The present invention is directed to methods for maintaining a desired shape of the cornea following the wear of corrective CKR™ contact lens(es), for example, reverse geometry contact lens(es) for an extended period of time post-wear. Typically, once the patient's cornea has achieve a desired reshaping and visual acuity, the patient may wear the corrective contact lens for a period of time, for example, 3-8 hours, typically overnight, and retain visual improvement and reshaped cornea for about 12-48 hours. As discussed in the examples below, administering to the CKR™-reformed cornea an effective collagen cross-linking amount of a cross-linking agent such as a photosensitizer (e.g., riboflavin) solution in combination with UV light effectively extends the amount of time the reformed cornea maintains a desired corneal shape and improved vision.

### CKR™

In the CKR™ method, a corrective contact lens is designed, fitted, and monitored using computer enhanced video-keratoscope to measure total corneal topography. A reverse-geometry lens, when applied to the cornea of a patient exerts a selective pressure on the cornea causing displacement of corneal tissue away from a zone of applied pressure to a zone of relief, thereby reshaping the patient's cornea and improving the patient's vision without surgical intervention. In general, the design of the mold induces change in the corneal topography of the patient's eye to make a myopic eye more oblate or a hyperopic eye more prolate.

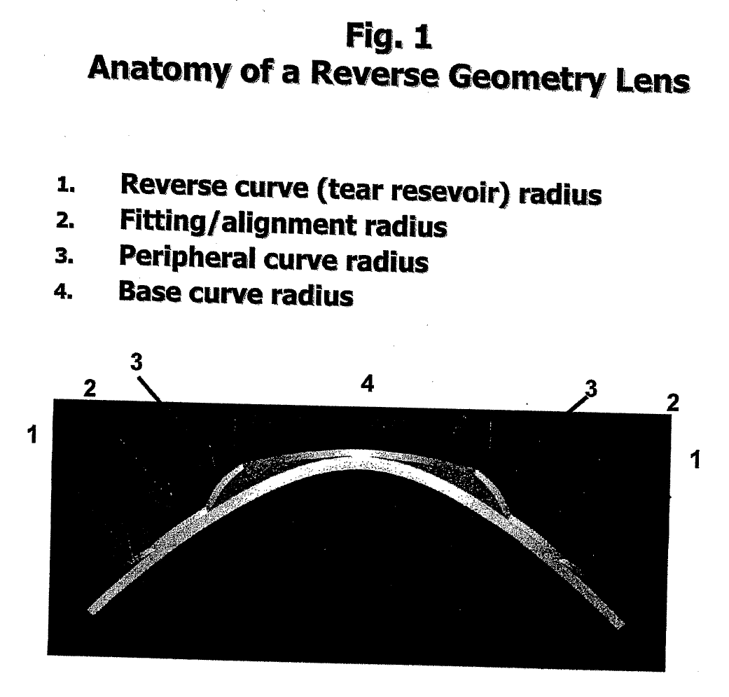

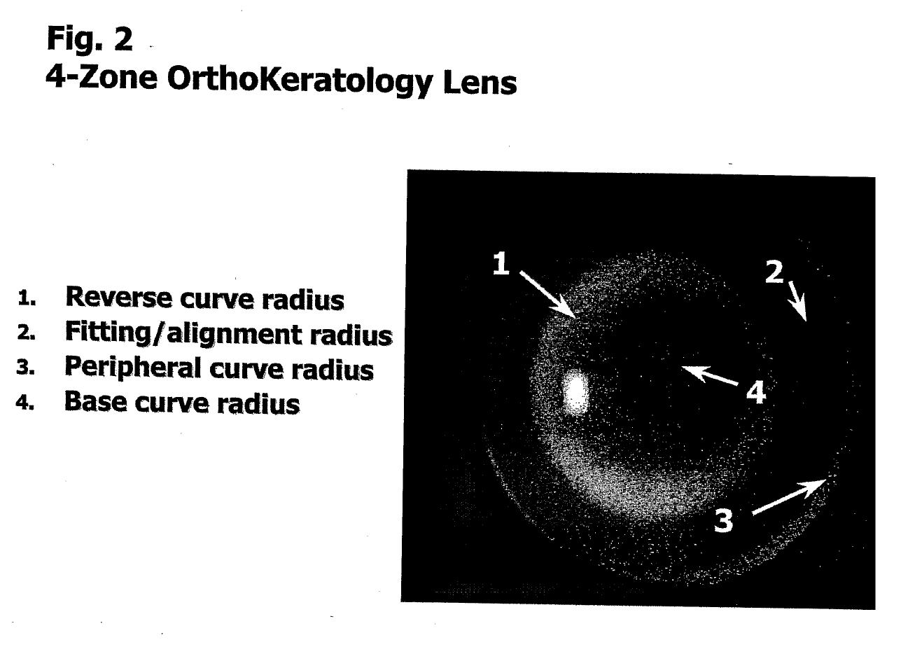

Reverse-geometry lenses (see, for example, FIG. 1) are tooled in response to the specific contour or topography of a patient's cornea and to affect a desired reshaping or correction of the eye's curvatures. As shown in FIG. 1, CKR™ reverse-geometry lenses have 4 curves, i.e. (1) a back optical zone radius (BOZR), (2) a reverse curve (RC), (3) an alignment curve (AC), and (4) a peripheral curve (PC). Centration of a reverse-geometry lens over the patient eye, as shown in FIG. 2, is important to success and good clinical outcome.

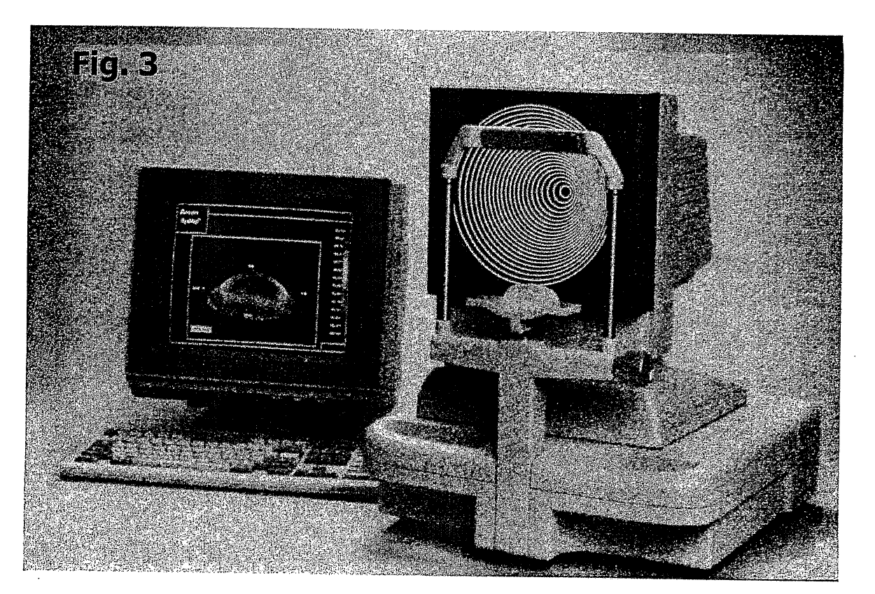

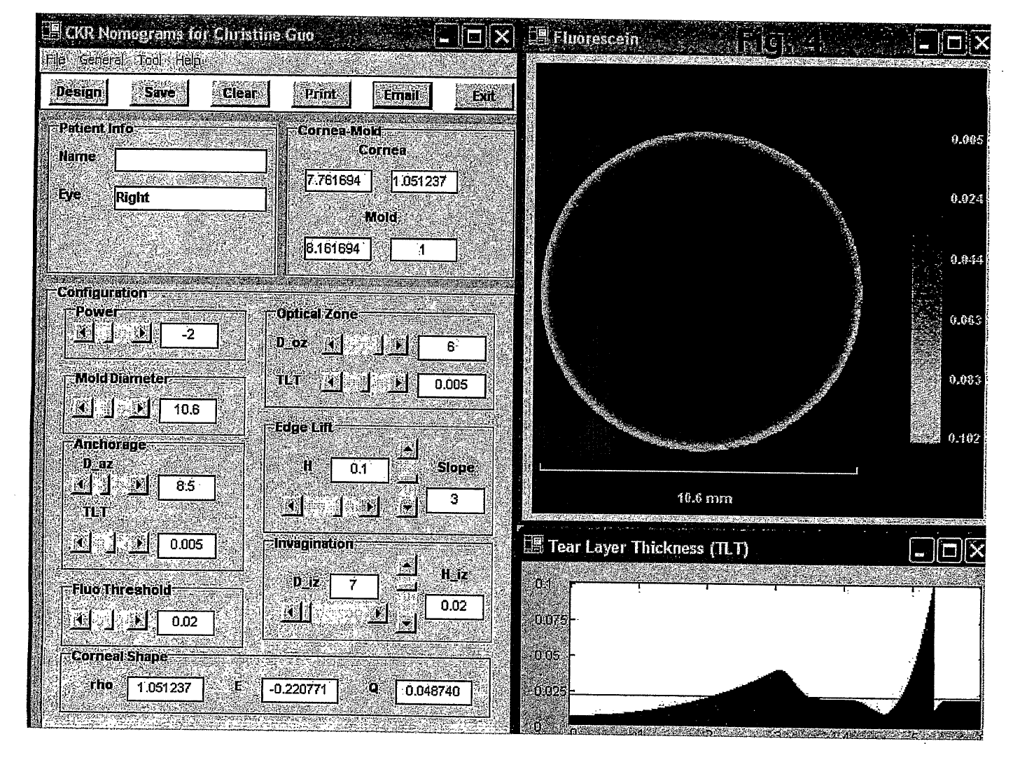

The orthokertology (CKR™) procedure includes fitting of reverse-geometry lenses that are designed with the aid of a computerized corneal topographer (FIG. 3). The design of the reverse-geometry lens is based on sagittal height, chord length, and shape factor of the cornea. As shown in FIG. 4, data from the corneal topography measurements are displayed on a computer screen, including the target myopia reduction, overall diameter of the reverse-geometry lens (OAD), back optical zone radius (BOZR), location of anchorage zone (AC), width and height of invagination (RC), back optical zone diameter (BOZD), peripheral curve (PC), and slope. FIG. 4 also shows the fluorescein pattern of the contact lens of the cornea, the tear layer thickness (TLT), the shape-factor (SF), eccentricity (e), and asphericity (Q) value.

Using the data obtained, CKR™ corrective lenses are designed and obtained, for example, using a CKR™™ fitting program that may be integrated with a lens design and manufacturing program such as Focal Points™, described in the Examples below. A high DK Boston material that is FDA approved for overnight wear such a Boston Equalens II™ oprifoconA (Bosch & Lomb) is preferred, to insure adequate oxygen supply to the cornea.

A reverse-geometry lens useful in the treatment of myopia contains a central pressure zone, an adjacent annular relief zone, and an annular anchor zone adjacent to the relief zone and located between the relief zone and the periphery of the reverse-geometry lens. When the reverse-geometry lens is positioned on the patient's cornea, pressure is exerted by the central pressure zone on the approximate center of the corneal dome, thereby effecting displacement of corneal tissue away from the center of the dome and to the adjacent annular relief area. The pressure exerted at the anchor zone controls reformations of the corneal surface by guiding the displaced tissue into the relief zone. With time, the steep curvature of the eye's corneal dome is flattened or reduced, and light incident over the central cornea will more correctly converge on the retina, thereby improving the patient's vision.

In the treatment of hyperopia, the pressure zone of the reverse-geometry lens is positioned to apply pressure at the approximate mid-periphery of the patient's corneal dome, and the adjacent relief zone is centrally located. When the reverse-geometry lens is applied to the patient's corneal surface, corneal tissue is displaced away from the mid-periphery and toward the relief area at the center of the dome, thereby increasing the steepness of the hyperopic eye's corneal curvature. The pressure zone of a reverse-geometry lens used in the treatment of hyperopia functions also as the anchor zone. The shape of the hyperopic eye is altered to more prolate shape, permitting incident light to converge on the retina, and thereby improve vision.

To effect treatment of astigmatism where the patient's corneal dome has more than one curvature, each at a given axis, the reverse-geometry lens's curvature places the pressure zone to apply pressure at the steepest meridian to effect its reduction and to minimize or eliminate differences in the curvatures. The characteristics of the reverse-geometry lens for treating astigmatism are similar to those for a mold for correcting myopia.

A typical CKR™ corrective contact lens will have, for example, a back optical zone diameter (BOZD) of about 6.00 mm, an overall diameter (OAD) of about 10.60 mm, an invagination (RC) of about 30 microns, an anchorage zone (AC) depending on the slope and the asphericity of the cornea, and a peripheral curve (PC) of about 90 microns. Typically, after a night's wear of the reverse geometry contact lens, the cornea changes for example, from a prolate ellipsoidal shape to an oblate ellipsoidal shape, that is, there is a shift from the steep side of the ellipse to the flat side of the ellipse.

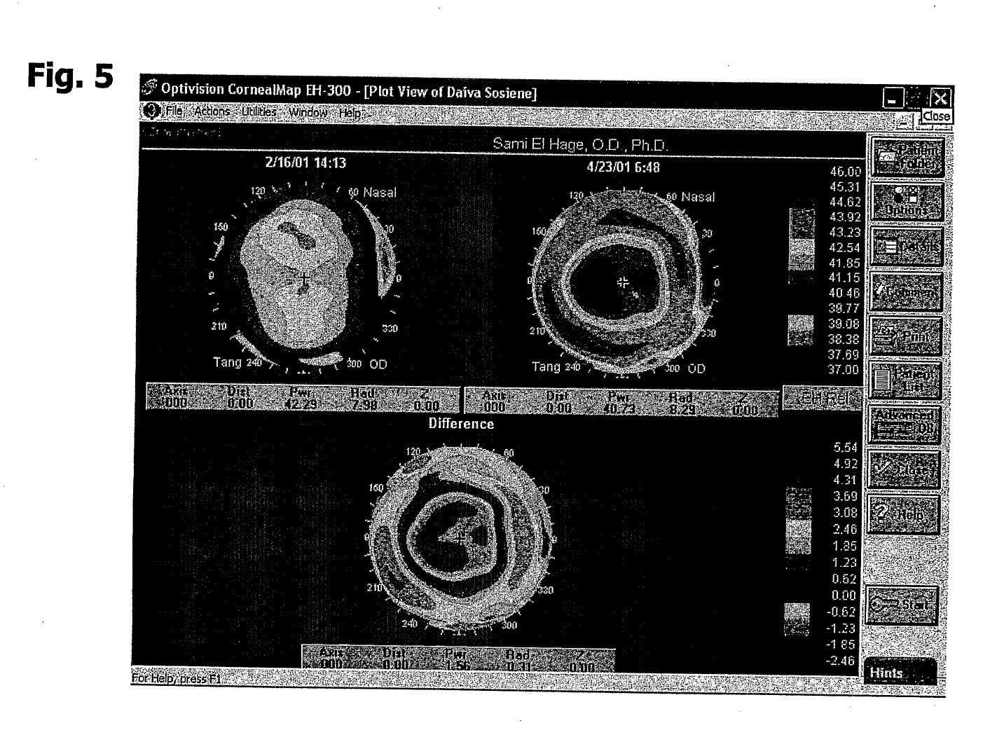

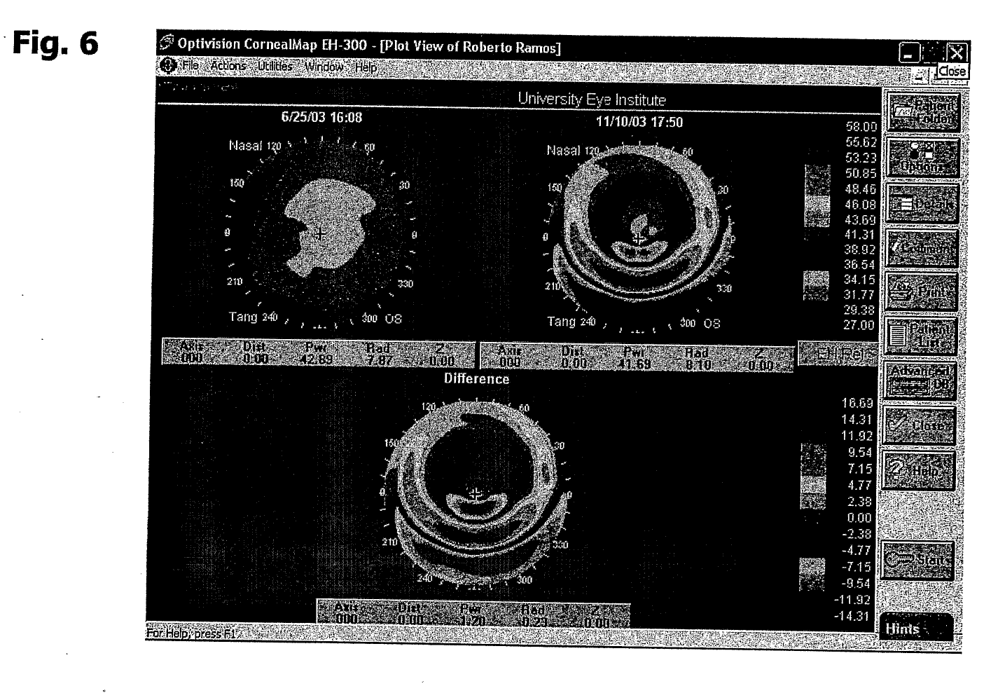

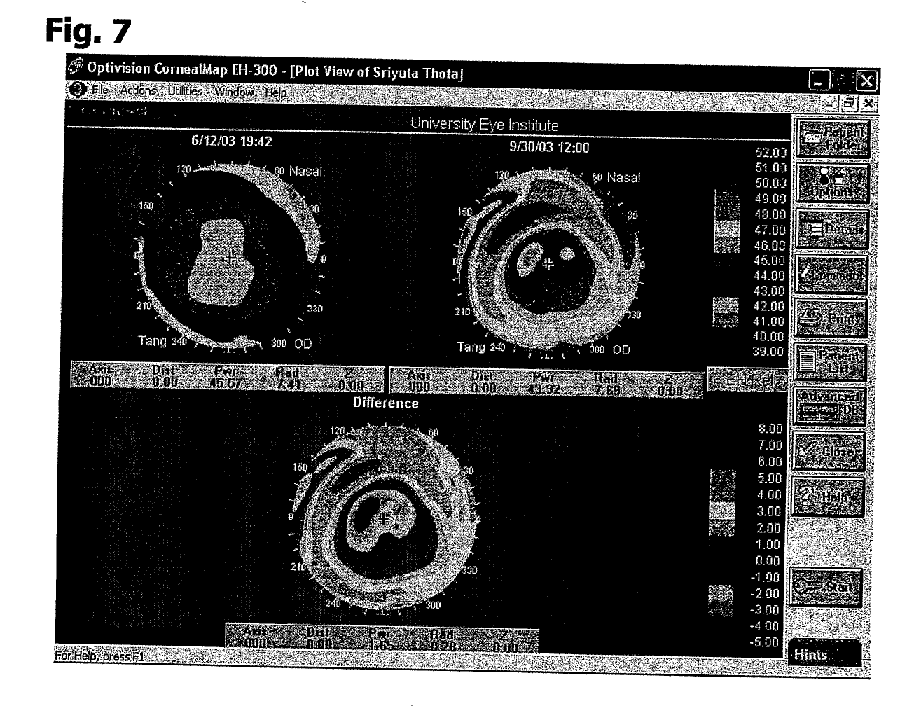

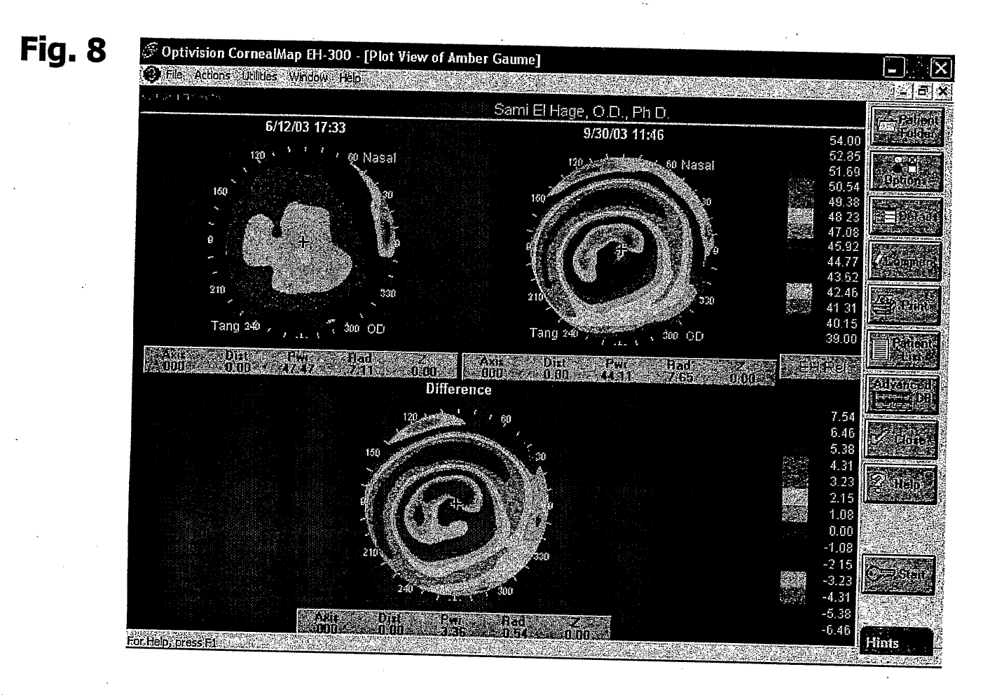

Corneal topography for centered reverse-geometry lens will show a nice central flattening of the cornea, followed by a steeper zone (the knee), and another flat zone to the periphery (see FIG. 5). If the reverse-geometry lens decentralizes while the patient is sleeping, a different corneal topography is noticed, which shows either an upper decentration of the reverse-geometry lens (in the case of an excessive flattening) (FIG. 6), a downward decentration of the reverse-geometry lens (FIG. 7), or a central island (FIG. 8).

### Patient Selection

Eye examinations and slit lamp observations are important in patient selection for the CKR™/crosslinking procedure. The cornea should not be too steep or too flat, and it is best if selected patients have a central reading between 40.00 D, and 48.00 D. Shape factor of the cornea should be below 1. Tear quality and quantity should be evaluated, and BUT should be evaluated using fluorescein and blue light along with yellow Wratten filter #11. Patients with dry eyes are generally not good candidates for the procedure. Depending on the severity of the dryness and the age of the patient, treatment, for example, Restasis (cyclosporine 0.05%) therapy can be initiated. Eyelids should be examined and special attention should be directed to blepharitis and meibomietis. If found, treatment should be initialized prior to the CKR™/crosslinking treatment.

### CKR™-C3R

As described more fully in the Examples below, application of a reverse-geometry lens to a patient's cornea in combination with corneal cross-linking, for example, using riboflavin and UV light, results in reshaping of the cornea and provides improved visual acuity that is maintained for a longer period of time than achieved by CKR™ in the absence of corneal cross-linking. It is appreciated that the reshaping of the cornea achieved by the combination methods of the present invention provides generally a more lasting, and potentially permanent result.

### Debriding

To ensure penetration of the corneal tissue on administration of the crosslinking agent, gentle debriding or cutting of the epithelium layer of the patient's eye is performed. The epithelium layer can be debrided from the cornea, for example, with a sponge, an alcohol solution of about 20% concentration, cutting with a scalpel, or any other known technique. The size of the debriding area can be, for example, about 4 to 9 mm, preferably about 5-8 mm, and most preferably about 5-6 mm in diameter. In one embodiment, the corneal epithelium may be debrided over an area approximately 7.8-9 mm. In another embodiment, vertical and horizontal cuts, for example, scalpel cuts, can be made in the epithelial layer. Two or more, for example, three vertical slits and one or more horizontal slit, of about 1 mm width and 4 or 5 mm length can be made on the epithelium layer to help the administered crosslinking agent such as riboflavin to diffuse throughout the cornea.

### Crosslinking Agent

A variety of crosslinking agents can be used to increase the rigidity of the cornea. One crosslinking-agent useful in the invention includes the combination of a photosentsitizer such as riboflavin with UV light sufficient to induce collagen crosslinking in the cornea. For example, at least about one drop of a photosensitizer solution such as a riboflavin solution is administered in a manner to permit the agent to penetrate at least ¾ of the corneal tissue, for example, at least 300 nm. Penetration of healthy corneal tissue leads to a better crosslinking and rigidity of the corneal tissue and longer-lasting maintenance of a desired corneal shape. Penetration can be enhanced, for example, by administering the agent to a debrided or scalpel cut area of the cornea.

The photosensitizer solution can be, for example, a solution of dextran and riboflavin, such as a dextran and riboflavin solution comprising 0.1% riboflavin (10 mg riboflavin-5-phosphate) and 10 mL 20% dextran-T-500. The photosensitizing solution is administered to the eye for a time sufficient to achieve penetration of at least about ¾ of the corneal tissue, for example, about 300 nm, and can be for about 10 to about 15 minutes.

In on embodiment, the photosensitizing solution is administered to the eye for a time sufficient for the riboflavin to reach the anterior chamber of the eye, and can be confirmed by viewing in a slit lamp. Viewing a cross-section of the anterior segment of the eye after administering the riboflavin solution, the concentration of the riboflavin solution in the cornea and the anterior chamber is sufficient to impart a yellow coloration to the cornea and to the anterior chamber. Preferably, the yellow coloration of the corneal tissue and in the anterior chamber is similar or the same. The slit-lamp observation helps to ensure the riboflavin solution reaches the anterior chamber of the eye, helping to protect the crystalline lens and the retina from small residual amounts of UV light.

The riboflavin-treated eye is preferably continuously irrigated during exposure to the UV light. The eye is preferably irrigated with a first irrigating solution every 1 or 2 minutes followed by a second irrigating solution every 1 or 2 minutes. The first irrigating solution is preferably a physiological serum (saline solution) and anesthetic (tetracaine 1% or other). The second irrigating solution is preferably a riboflavin solution. The riboflavin solution is preferably 0.1% riboflavin (10 mg riboflavin-5-phosphate) and 10 mL 20% dextran-T-500.

### UV Light Exposure

The riboflavin treated cornea with is exposed to UV light, for example, using an LED radiating light at about 360 to about 370 nanometers, preferably about 365 nm, with an intensity of about 3 mW/cm². The UV exposure is generally for at least about 30 minutes, but can be more or less, as needed.

Upon completion of the UV treatment, the treated eye is observed again using the slit-lamp to check the health of the eye. For example, the cornea is observed to determine if there is a haze or not, the anterior chamber is checked to determine if there is any flare, and the crystalline lens is checked.

After the crosslinking procedure, the method the patient may be fitted with soft contact lenses. The soft contact lenses in this case are used as a bandage to protect the eye. The patient may also be prescribed a non-steroidal anti inflammatory agent, such as ACULAR, ALREX OR LOTEMAX, for example, optionally with an antibiotic such as ZYMAR, VIGAMOX OR ZYLET, and/or a pain killer.

The procedure further includes following-up with the patient the day after the procedure, 3 days, one week, one month, 3 months, 6 months, and one year after the procedure including a routine eye examination.

### EXAMPLES

The invention may be further understood by reference to the following Examples. The Examples are not intended to limit the scope of the invention, but serve to exemplify specific embodiments.

### Example 1

### Validation of CKR™ Procedures

Reverse geometry gas permeable (GP) contact lenses when worn at night will modify corneal curvature resulting in the temporary improvement of unaided visual acuity in low to moderate myopes. Traditionally, these designs have characteristically required the use of diagnostic lenses to determine the best fit for a given wearer. The purpose of this study was to determine the efficacy of fitting advanced orthokeratology lenses, Controlled Kerato Reformation (CKR™™), empirically from corneal topography data without and/or with the use of diagnostic lenses.

The efficacy and ease of empirically fitting gas permeable (GP) contact lenses, manufactured in Boston Equalens II (oprifocon A) material, for orthokeratology (CKR™™) was examined. At the time of this study, the Boston Equalens II (oprifocon A) Dk=85×10-11 (cm³O₂(cm/[sec)(cm2)(mmHg)] @ 35° C. material was marketed with Food and Drug Administration (FDA) approval for both daily and extended wear conventional GP lenses and for daily wear orthokeratology lens designs. In June 2004, Boston Equalens II material was approved additionally for overnight orthokeratology.

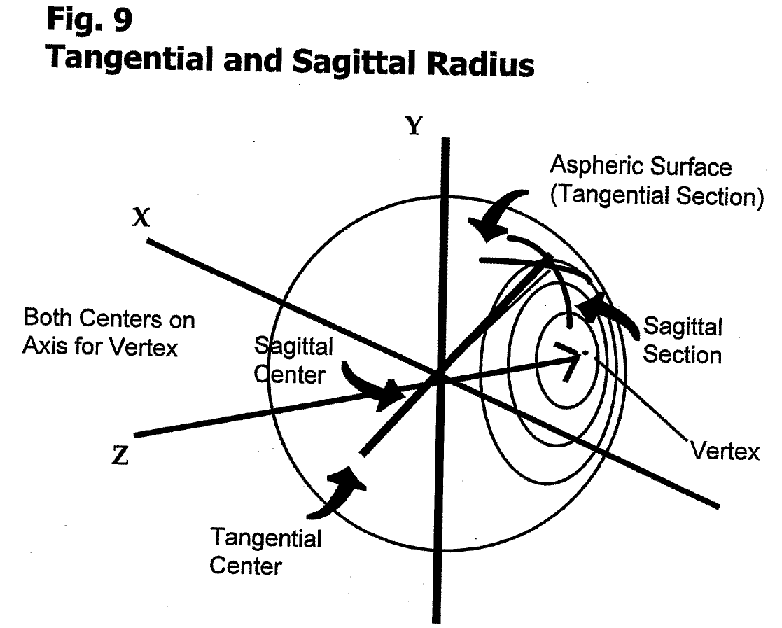

In this clinical trial we used an approach of designing and fitting orthokeratology contact lenses that was based on corneal topography and tear layer thickness (TLT). The corneal topographer used provided three coordinates (x,y,z) of each point on the cornea (see FIG. 9), allowing reconstruction of the shape of the cornea and design of the lens from corneal topography information utilizing a built-in software program.

Another consideration is the tear layer thickness (TLT) between the contact lens and the cornea. In normal GP lens designs, there is a tear thickness of about 20 microns between the cornea and the back surface of the lens. This concept is even more important in fitting reverse geometry contact lenses in orthokeratology. A change of a few microns in the cornea to contact lens relationship, for example, in the central segment of the lens, may induce a diopter or more in refractive change. A change of only a few microns in the return curve segment or in the alignment segment, will affect the centration of the contact lens over the cornea.

For instance, in order to center the contact lens over the patient's cornea, increasing the return curve depth of a few microns may be needed to tighten the fit. Alternatively, to loosen the lens, the return curve depth may be decreased. The same applies to the alignment curve. By varying the slope of the alignment curve we can bring it closer or farther away from the cornea. Bringing the alignment curve closer to the cornea will tighten the fit of the contact lens, and moving the alignment curve away from the cornea will loosen the fit.

In this study, the CKR™™ fitting program was integrated with Focal Points™ Lens Design to design and manufacture the lenses. After designing the orthokeratology lens from the computer nomogram, the lens order was sent to the laboratory electronically. The expected benefit of such an empirical system simplifies the overnight therapy process of achieving temporary improvement in unaided visual acuity better than or equal to 20/40 while yielding a clinically acceptable physiologic response.

Modern orthokeratology requires, at minimum, to fit the lens according to sagittal height and corneal asphericity, eccentricity or shape factor, and at best is based on corneal topography. The traditional notion of fitting contact lenses based on K, for example, flatter than K or steeper than K, is not applicable in modern orthokeratology. K is the central keratometric reading of the cornea and assume the cornea to be a sphere.

Corneal topography is generally accepted as the standard of care in orthokeratology for follow-up and assessment of the fitting and positioning of the contact lens over the cornea, and the health of the cornea. Along with visual acuity and over refraction, corneal topography provides the best indication of the efficacy of the procedure.

### Methods

Thirty-two subjects were screened and twenty-nine subjects were entered into the study (11 males and 18 females). Nine subjects withdrew or were discontinued for various reasons. The average age of the 20 subjects completing the study was 26 years±3.7 years, ranging from 20 to 32 years. Females comprised 73% of the cohort and males 27%.

The protocol was approved by the University of Houston Committee for the Protection of Human Subjects prior to initiation of the study. Each subject was examined at the initial visit to determine eligibility. To be enrolled into the study, subjects were required to have normal ocular and systemic health, myopia of between 1.00 and 4.00 diopters (D), astigmatism no greater than 1.50 D, and no previous history of GP lens wear. The study was explained to the subjects and they were asked to read and sign a statement of informed consent.

Once enrolled, corneal topography (Optivision CornealMap™), measurements were obtained, study lenses were ordered, and the subject was scheduled for a dispensing visit. The design of the lenses was determined directly from corneal topography data and the CKR™™ computerized fitting nomogram utilizing Focal Points™, without the use of diagnostic lenses. The values of shape factor (SF), eccentricity (e) and asphericity (Q) were shown in the software program for each cornea, as well as the fitting nomogram. The CKR™™ nomogram demonstrates the lens characteristics, the fluorescein and the tear layer thickness under the contact lens (TLT). This program produces an aspheric back surface orthokeratology lens design.

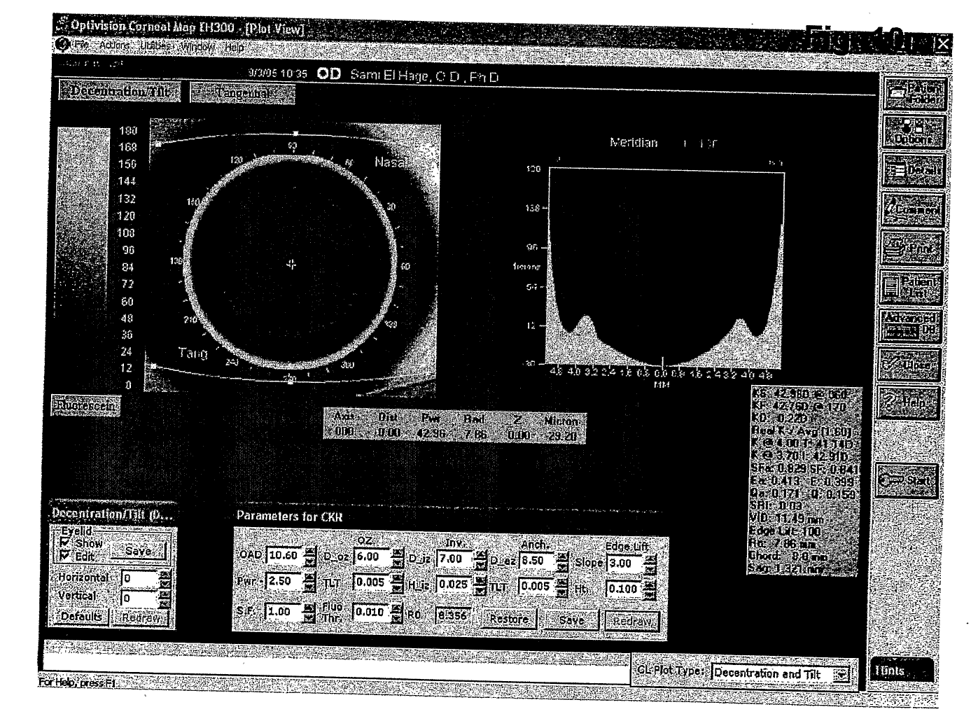

FIG. 10 shows the CKR™™ fitting nomogram as it appears on the computer monitor. Displayed on the left hand side is the simulated fluorescein pattern (“bulls eye”) showing the lens to cornea fitting relationship of the designed contact lens. On the right hand side is the tear layer thickness (TLT) profile between the contact lens and the anterior corneal surface. The software allows indication of the amount of tear thickness required at the center of the cornea versus the amount required at the junction between the return curve and the alignment curve. The lower left side allows for simulated horizontal and vertical movement of the contact lens over the cornea. Finally, the lower right side of the CKR™ nomogram shows the parameters of the designed contact lens, i.e., the overall diameter (OAD), the optical zone (OZ), the amount of aimed myopia reduction (Pwr) the shape factor of the lens design (SF), the depth and width of the invagination or reverse curve (Inv), the anchorage (Anch) or alignment zone and its slope, and the edge lift.

The computer software allows customization of any of these parameters to achieve an optimally fitting lens. For example, by changing the slope of the anchorage zone, the edge lift will increase (loosen) or decrease (tighten) affecting the lens position. Likewise, the location of the alignment curve and the diameter of the lens may be customized to influence the fit.

At the right side of the nomogram the values of the K-readings, corneal astigmatism (KD), shape factor (SF), eccentricity (e), asphericity (Q), central radius of curvature (Rₒ), the surface regularity index (SRI), visible iris diameter (VID), edge lift, chord and sagittal height are shown. The asphericity of the cornea is expressed in terms of shape factor (ρ), eccentricity, (e) or asphericity (Q), where ρ=1−e² and Q=−e². After the lenses were designed, the data was electronically sent to the laboratory via the internet, where the lenses were made.

In addition to the basic eye examination and measurements of corneal topography, confocal microscopy (Nidek, Inc. ConfoScan3), Shack-Hartmann aberrometry, ultrasound corneal thickness measurements (Sonogage CorneaGage Plus) and scanning slit topography/corneal thickness (Orbscan II) data were collected. At the initial dispensing subjects were asked to wear the lenses overnight and follow up visits were scheduled at one day, one week, two weeks, one month, three months and six months.

Corneal topography, subjective refraction, and high contrast aided and unaided logMAR visual acuity were measured at each office visit. LogMAR visual acuity is a logarithmic transformation of an optotype's size in minutes of arc that creates a geometric progression and simplifies statistical analysis. Each subject was also asked to complete a questionnaire regarding lens comfort and their unaided visual acuity when performing certain tasks. Scanning slit topography/corneal thickness, confocal microscopy, pachymetry and aberrometry were taken prior to fitting and then at the one month, three month and six month follow up visits. During the test period, patients wore the CKR™ lenses approximately 8 hours per day (overnight), and removed the lenses during the daytime.

### Results

During the 6-month study, unaided logMAR acuity improved from 0.78±0.26 OD/0.75±0.22 OS to 0.06±0.18 OD/0.04±0.16 OS. Myopia was decreased from −2.55 D±0.87 OD/−2.47 D±0.89 OS to +0.45 D±0.74 OD/−0.17±0.69 OS. Shape factor, as determined by corneal topography, increased from 0.85±0.13 OD/0.85±0.15 OS to 1.28±0.32 OD/1.30±0.29 OS. Both eyes demonstrated a decrease in lower order aberrations (defocus) and an increase in higher order aberrations (spherical aberrations and coma). Neither total nor epithelial corneal thickness as measured by ultrasonic pachymetry, scanning slit topography, and confocal microscopy varied significantly from baseline measurements.

All data were analyzed by a mixed effect repeated measure analysis of variance (ANOVA). In the mixed effect model all the data is used to compute the correlation matrix and standard deviations. Thus, all eyes were used without averaging. SAS Proc mixed (the computer subroutine) corrects for correlations between eyes and unnecessary gains in power due to repeated measures over time. To correct for significance due to multiple comparisons, the Tukey-Kramer Correction was applied in all calculations.

### Visual Acuity

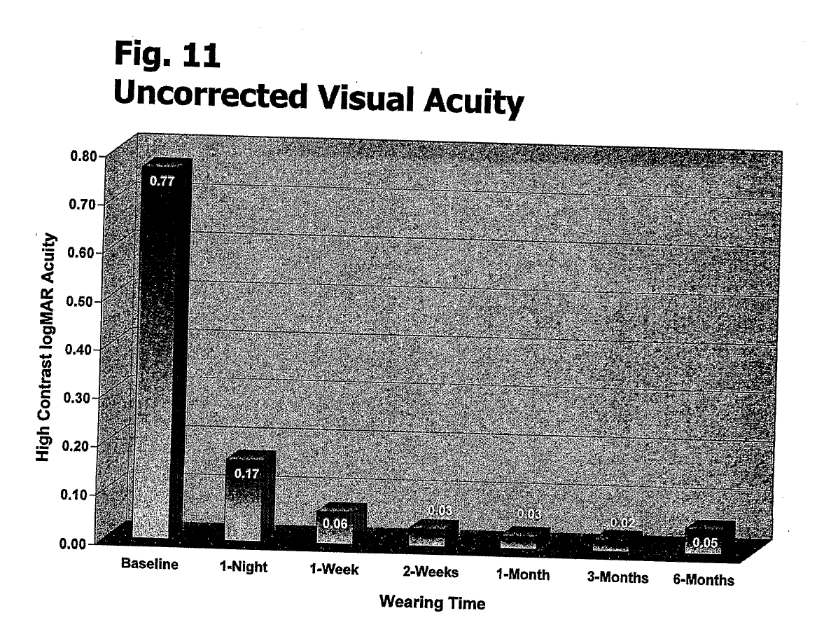

Baseline visual acuity values for each patient were compared to each subsequent visit. When compared to baseline, the t-value for each visit showed a significant change (p<0.0001). Over the seven time points in which vision without correction was assessed there was a significant difference (F=59.99, p<0.0001). An initial improvement in unaided high contrast logMAR visual acuity was noted after the first night of lens wear and the majority of subjects reported functional vision for most of the day. The peak of visual acuity improvement was reached at the end of the first week as seen in FIG. 11.

### Refractive Error

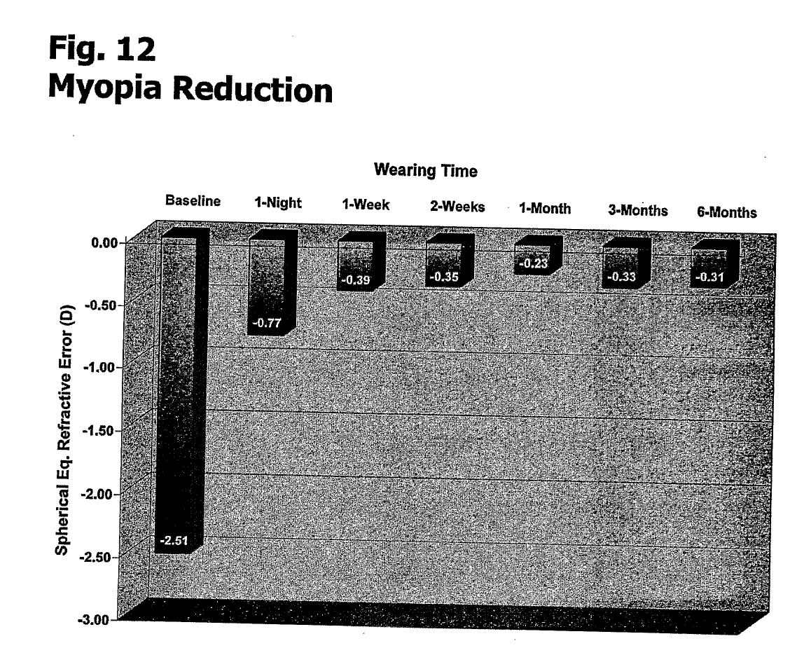

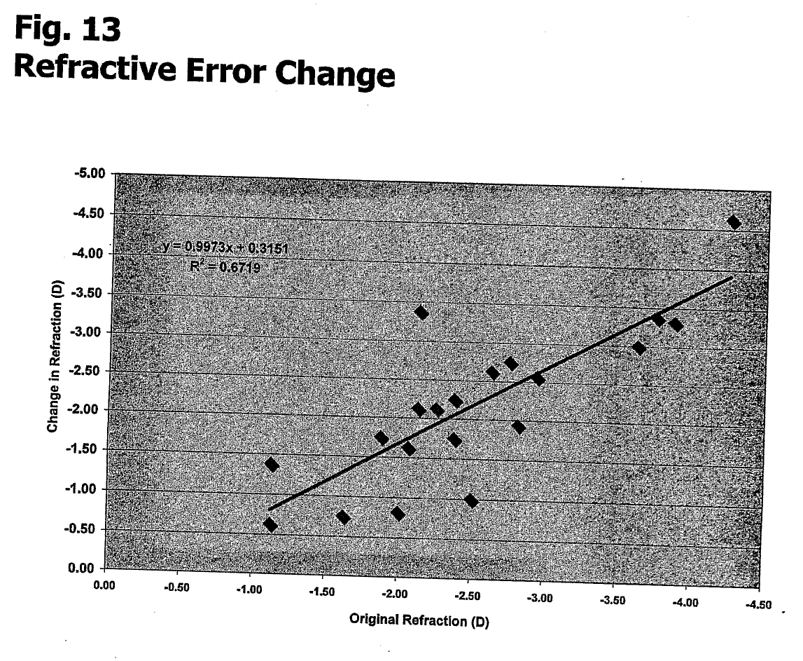

Subjects achieved a 66% to 80% reduction in refractive error (FIG. 12). Baseline sphere values were compared to each subsequent visit. When compared to baseline the t-value for each visit showed a significant change (p<0.0001). Dioptric sphere power changed significantly over time (F=36.30, P<0.0001) from −2.04 D to −0.33 D at 180 days. On the first overnight follow-up visit the mean refractive error was −0.72 D and −0.36 D by day 14. The change in refractive error as a function of the baseline refractive error is displayed in FIG. 13.

### Keratometry

Keratometry in the horizontal axis did not change clinically or statistically over time and follow-up visits did not differ from baseline values. However, keratometry change in the vertical axis were statistically significant over time (F=5.33, P=0.002). Comparisons from baseline showed small, clinically insignificant changes (data not shown).

### Corneal Topography

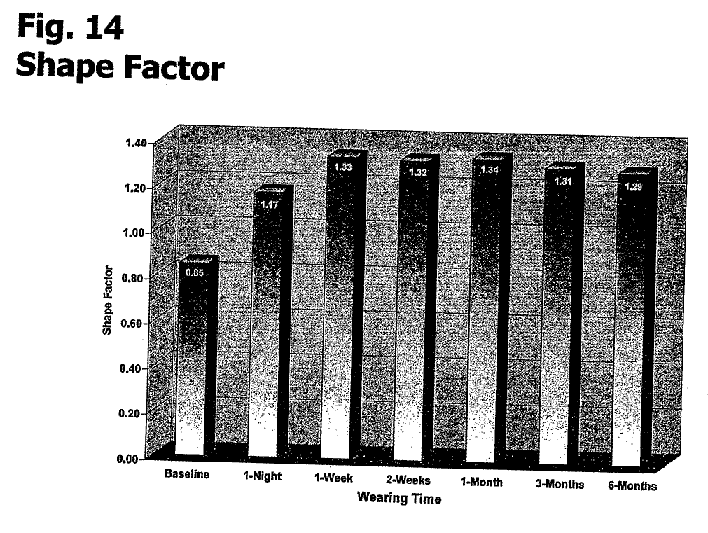

Corneal topography, as determined using the Optivision CornealMap™, was shown to change significantly over time (F=21.21, P<0.0001). When compared to baseline, change was significant at each subsequent visit, after correcting for multiple comparisons. The shape factor (SF) demonstrated a significant change (F=13.78, P=0.0003) with the average shape factor increasing in proportion to the change in refractive error from a prolate to oblate ellipsoidal shape (FIG. 14). When the shape factor increased by one unit, the sphere equivalent decreased by 0.8153 (±0.1412). Also, when the shape factor increased by one unit, the logMAR results improved by 7.58 (±2.04) letters or by a gain greater than one line of vision on the Bailey-Lovie Chart.

### Higher Order Aberrations

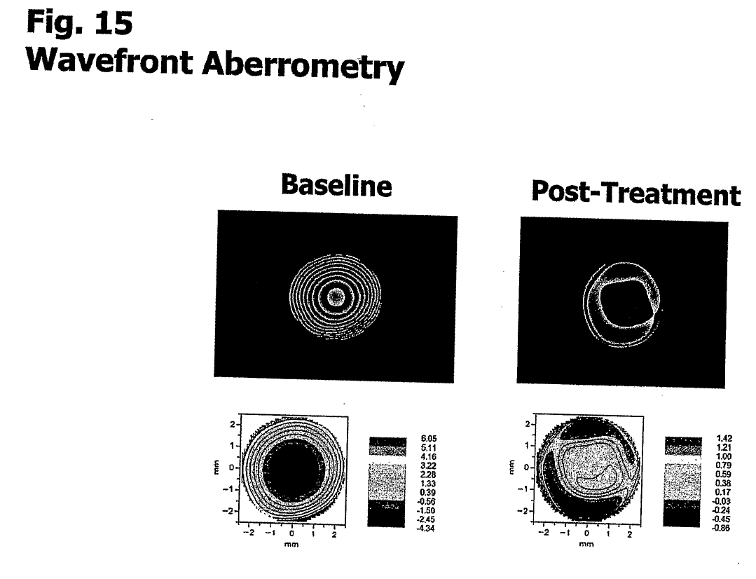

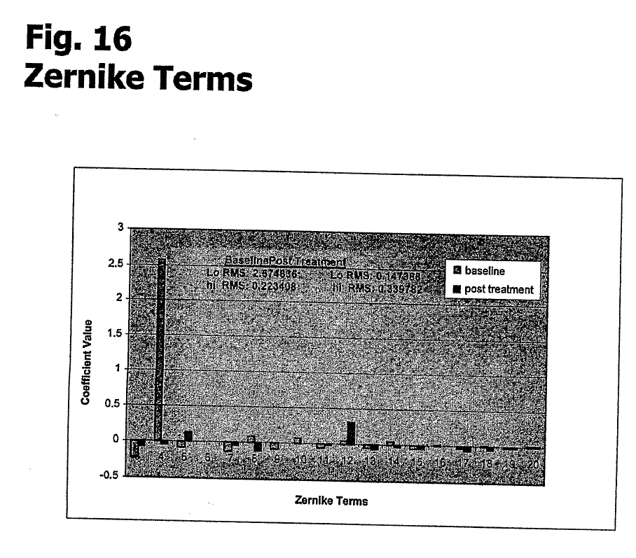

Higher order aberrations were determined using an in house Shack-Hartmann aberrometer. Measurements were taken in total darkness without pupil dilation and the Zernike polynomials were calculated for a 4 mm pupil diameter. A decrease in lower order aberrations, primarily defocus, with an increase in higher order aberrations, primarily coma and spherical aberration occurred. See FIGS. 15-16. These results were not unexpected and are similar to those found in post-refractive surgery. As with refractive error, defocus and higher order aberrations, did not change after the one month visit.

### Confocal Microscopy

The Nidek ConfoScan3 Confocal microscope is a non-invasive instrument that images the cornea. It is a commonly used instrument in highly specialized corneal practices for the express purpose of diagnosis, especially diseases of the cornea. Each cornea was imaged in nearly 60 seconds with 350 images gathered throughout the entire corneal thickness at 5 micron intervals. Inspection of the Confocal microscopy images indicated no significant morphological changes secondary to orthokeratology.

### Corneal Thickness

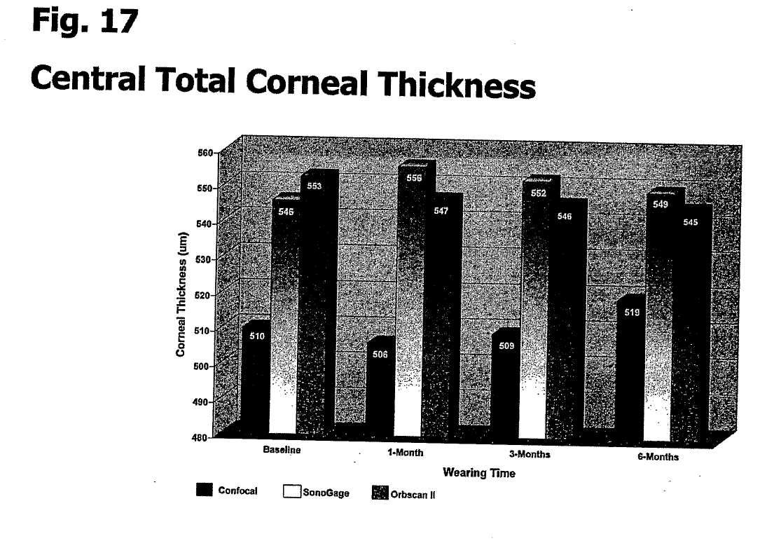

Corneal thickness measurements were taken using three different instruments: The Nidek ConfoScan3, the Sonogage CorneaGage Plus and the Bausch and Lomb Orbscan II. Data are show in FIG. 17. The CorneaGage Plus, according to the manufacturer, can measure both total and epithelial corneal thickness while the Orbscan II is capable of providing a profile of total corneal thickness across the corneal surface. The ConfoScan3 instrument is not generally used for obtaining corneal thickness measurements, but was said to have that capability.

The total central corneal thickness (TCCT) was measured using Orbscan II, ConfoScan 3, and CorneaGage Plus instruments. The Orbscan II did not detect any significant thickness change over time nor did it detect differences from baseline and subsequent visits. Total central corneal thickness was unchanged with this instrument. The confocal TCCT data are similar to that of the OrbScan II and did not show any thickness change over time nor did it detect differences from baseline and subsequent visits. Total central corneal thickness was also unchanged with this instrument. The CorneaGage Plus TCCT did not show thickness change over time nor did it detect differences from baseline and subsequent visits. Total central corneal thickness was unchanged as detected with this instrument as well.

Total inferior corneal thickness (TICT) was also measured with each instrument. The Orbscan TICT did not show thickness change in the inferior cornea over time nor did it detect differences from baseline and subsequent visits. Inferior corneal thickness was unchanged with this instrument. The Confocal TICT demonstrated a barely significant inferior thickness change over time, but did not detect differences from baseline and subsequent visits. Values changed by as much as 50 microns, but the change was random over time and did not show a consistent trend. The Sonogage instrument demonstrated a significant inferior thickness change over time (F=7.92, P<0.0001), but only detected differences from baseline and subsequent visit at 90 days post-treatment. Values changed by as much as 28 microns, but the change appears to be random over time and does not show a consistent trend.

When measuring central epithelial thickness (CET), the ConfoScan3 showed a barely significant epithelial thickness change over time by 10 microns and only detected a difference from baseline at the 180 day visit. There seemed to be a decreasing trend, but probably not clinically significant given test-retest variability. The CET with CorneaGage Plus showed a significant epithelial thickness change over time (F=7.69, P<0.0001) by only 1 micron and only detected a difference from baseline at the 30 day visit.

The inferior epithelial thickness (IET) with ConfoScan3 failed to show a significant epithelial thickness change over time, with just 5 microns variability, and did not detect a difference from baseline and any subsequent visit. The IET with the Sonogage instrument also failed to show a significant epithelial thickness change over time, with less than 1 micron variability, and did not detect a difference from baseline and any subsequent visit.

### Subjective Comfort

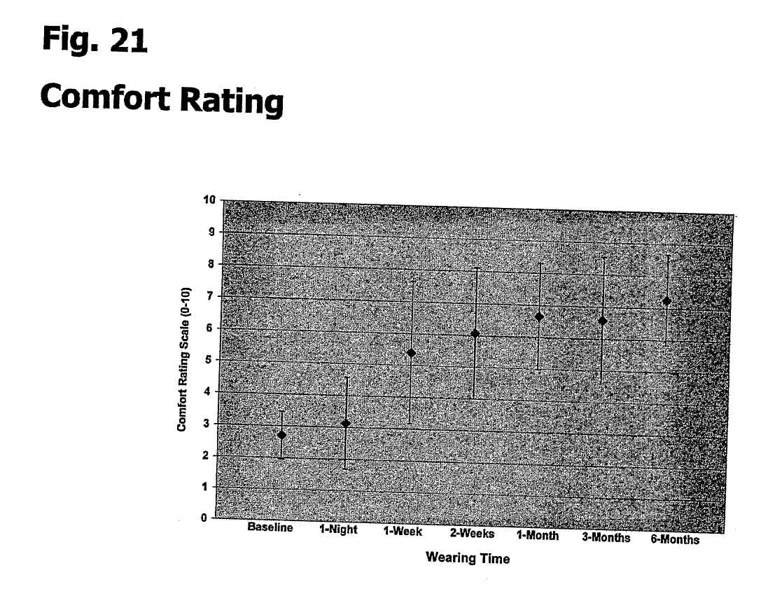

Subjects were asked to rate the overall comfort of the lenses on a scale of 0-10 with 0 being not tolerable and 10 being unable to feel. Comfort is a subjective estimate and in this study there was no control lens, thus there could be a bias toward patients giving information to please the investigators (the halo effect). However, there was a large change (F=75.01, P<0.0001) in perception of comfort over time from a comfort rating of 2.73 at the initial dispensing visit to 7.30 at visit 180 (FIG. 21).

### Conclusion

The CKR™™ empirical lens design was demonstrated to be an effective and safe method for temporarily reducing myopia and improving unaided visual acuity. The amount of myopia reduction found at the one week visit was clinically insignificant from the one month results indicating that the full effect is achieved by one week. There was an increase in higher order aberrations, primarily coma and spherical aberration (third and forth order of Zernike polynomial), with a decrease in lower order aberration, primarily defocus (second order aberration).

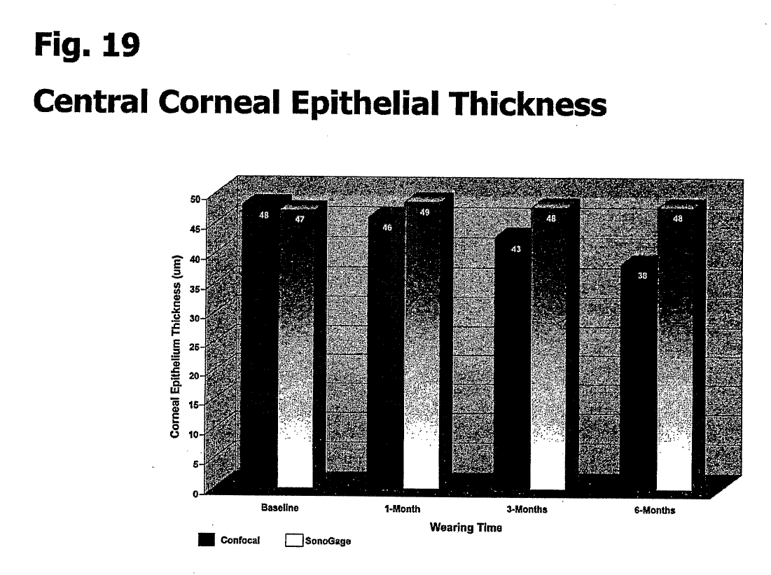

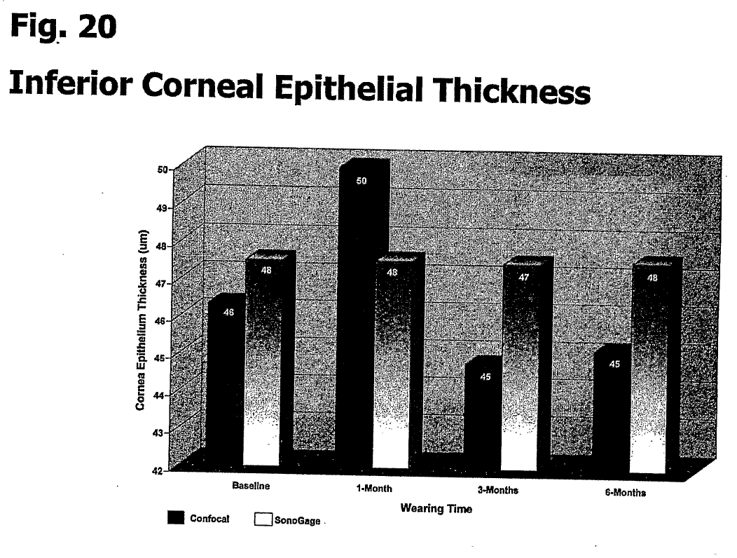

Changes in total and epithelial cornea thickness measurements using different instrumentation, is contrary to the results of many other researches. Both epithelial and total corneal thickness measurements showed variations as a function of the instrumentation used and the time of measurements. For instance, the B&L Orbscan II system showed different central corneal thickness results than the Sonogage CorneaGage Plus and the Nidek ConfoScan3. The ConfoScan 3 results showed a small decrease in central corneal epithelial thickness and a slight increase in peripheral epithelial thickness noted at the 3-month visit (FIGS. 19 and 20). The ConfoScan 3 showed a greater level of variability in epithelial thickness measurements while the CorneaGage Plus did not. Data are similar for each instrument used in assessments of corneal thickness. In view of the above results, no clear differences in total or epithelial corneal thickness were seen over time.

The amount of myopia reduction found at the 1-week visit was clinically insignificant from the 1-month results indicating that the full effect is achieved by one week. Neither total nor epithelial corneal thickness measurements show any statistically significant changes from baseline. These results demonstrate the effectiveness of the CKR™ lens design at reducing myopia and improving unaided visual acuity.

### Example 2

### Crosslinking of CKR™-Corrected Cornea

Corneal collagen crosslinking with Riboflavin (C3-R) has been shown to strengthen weak corneal structure in patients with keratoconus. A study was undertaken to determine if corneal crosslinking could be used to sustain the reformation of corneal shape and visual acuity achieved by CKR™.

In the crosslinking procedure, fresh, custom-made riboflavin drops are applied to the cornea, with or without debriding the epithelium. The riboflavin in the cornea is then activated by ultraviolet light. This treatment increases the amount of collagen crosslinking in the cornea, and thereby increases the biomechanical rigidity of the cornea.

In this study of six patients, 5 female, one male, average age 25 years, the patients had each been treated by CKR™ as described for Example 1 and had been wearing the CKR™ corrective lenses overnight, on average for 1.5 years. (Range of 12 months to 24 months) The average myopia in SE was −3.0 D, and average Shape Factor was 0.736.

Patients wearing overnight CKR™ corrective contact lenses came to the clinic the next morning wearing the lenses. Lenses were then removed. Visual acuity measurements, slit lamp observation with fluorescein evaluation, corneal topography measurements, post-refraction, and pachometry measurement of corneal thickness were obtained as described above for example 1. After confirming good functional visual acuity, the crosslinking treatment was begun with the patient in a supine position. An anesthetic, Tetracaine 1% solution was instilled in the cul-de-sac of the eye to be treated, in order to numb the cornea. Two lid separators were used to separate the superior and inferior lids. The epithelium was gently debrided using a sponge to separate and remove a portion of the epithelium layer from the cornea over an area of about 7.8 to 9 mm diameter. A solution of riboflavin mixed with dextran (0.1% riboflavin, vitamin B2, photosensitizer solution, 10 mg riboflavin-5-phosphate in 10 ml 20% dextran-T-500) was instilled in the eye under treatment. The solution was used on the eye for about 10 to 15 minutes, or until the riboflavin reached the anterior chamber of the eye, as confirmed by observation using a slit lamp and viewing a cross section of the anterior segment of the eye. The presence of riboflavin in the anterior chamber of the eye protects the crystalline lens and retina from a small residual amount of UV (about 0.1%) that could penetrate beyond the anterior chamber.

The patient's cornea was exposed to UV when the concentration of the riboflavin in the cornea and in the anterior chamber was the same or similar, as evidenced by the same or similar coloration of the cornea and the anterior chamber seen through the slit lamp. UV treatment was accomplished using an LED radiating light at 365 nm with an intensity of 3 mW/cm². The duration of the UV exposure was approximately 30 minutes. During the exposure, the patient was asked to look at the light source while the eye was irrigated with a combination of saline solution and anesthetic (tetracaine 1%) every 1 to 2 minutes, followed by irrigation of riboflavin solution every 1 to 2 minutes. After exposure to UV light, the patient was observed again using the slit lamp to confirm health of the eye, the cornea (e.g., no haze), the anterior chamber (no flare), and the crystalline lens.

After the procedure, patients were fitted with soft contact lenses, used as a bandage to protect the eye. A non-steroidal inflammatory agent (Acular) and antibiotic (Zymar of Vigamox) was administered drop wise. The patients were instructed to administer these medications four times a day for one week, and to administer pain killers if and as needed.

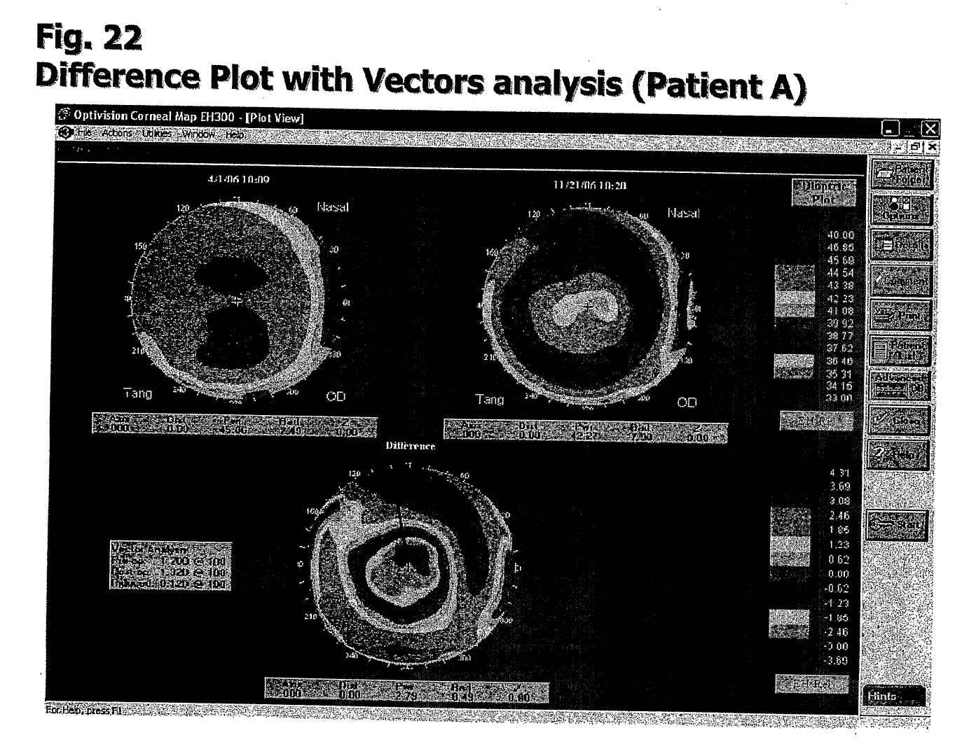

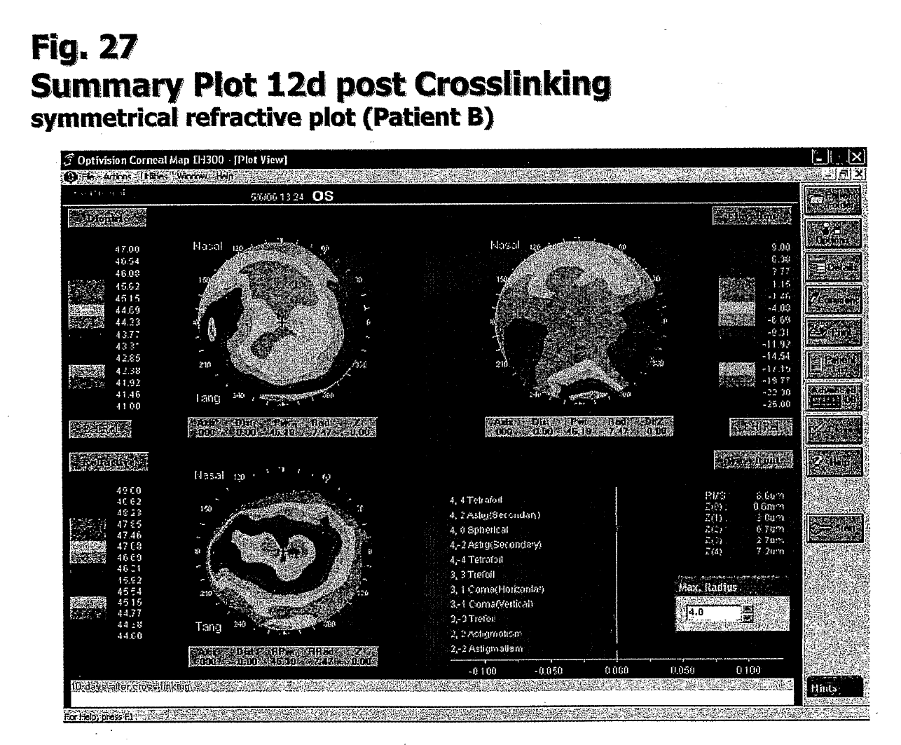

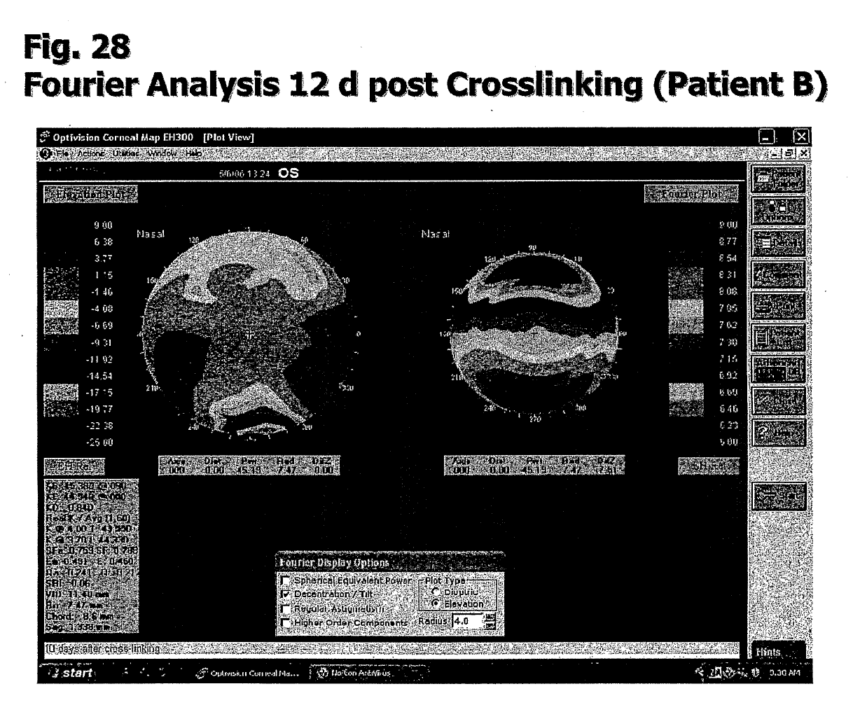

The results of the study showed an overall reduction in Shape Factor in the treated corneas, and changes in corneal rigidity (R) factor. The combined treatment also resulted in improved corneal symmetry, improved Tilt, first Harmonic in Fourier analysis. Some increase in high order aberration was also noted. The correction of corneal shape as well as improved visual acuity achieved by daily wear of the CKR™ corrective contact lenses was extended to longer-lasting, perhaps permanent correction by cross-linking of the corrected cornea, as analyzed in patients up to three months post treatment. At three months, all patients were now wearing a corrective CKR™ Lens only about one night per week to maintain improved vision. Data for specific patients are shown in FIGS. 22-36.

Representative Patient A

Changes in corneal topography pre and post CKR™ treatment for representative patient A are shown in the Optivision CornealMap™ displayed in FIG. 22. This Difference Plot shows the corneal topography of the cornea prior to CKR™ treatment (top left) and after more than 7 months of overnight wear of a CKR™ lens (top right). The color scale shows the relative dipotric plot, and the differences are shown (bottom). Refractive error was reduced −2.79 D and the dioptic conversion of the radius of curvature was increased by 0.49 mm, with no distortion or induced astigmatism.

### Representative Patient B

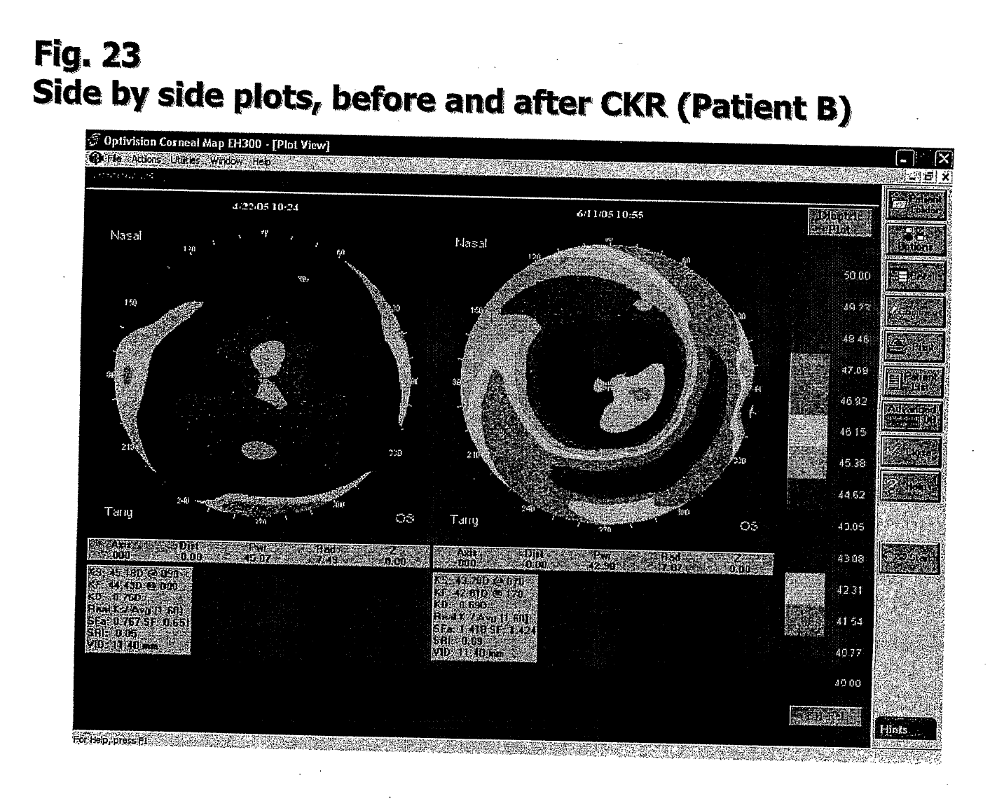

Changes in corneal topography pre and post CKR™ treatment for representative patient B are shown in the Optivision CornealMap™ displayed in FIG. 23. The side by side Plots show corneal topography prior to CKR™ treatment (left) and after almost 2 months of overnight wear of a CKR™ lens (right). The color scale shows the relative dipotric plot, and the differences are shown (bottom). Refractive power at the center of the cornea decreased from 45.07 D to 42.90 D (−2.17 D) and the radius of curvature increased from 7.49 mm to 7.87 mm (0.38 mm). The Shape Factor was increased from 0.651 to 1.424, i.e. from prolate to oblate side of the ellipse.

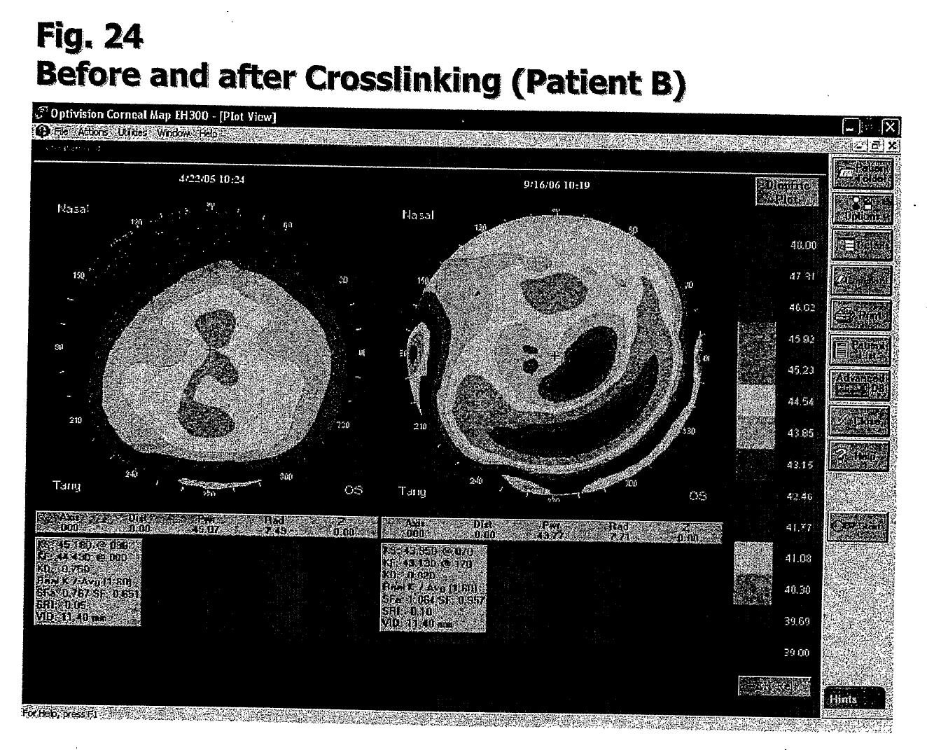

Changes in corneal topography after the crosslinking procedure for representative patient B are shown in the Optivisional Corneal Map™ displayed in FIG. 24. The side by side Plots show corneal topography prior to CKR™ (left) and post crosslinking (right). Comparing the data shown in FIGS. 23 and 24, the changes across the combined treatment are demonstrated:

| | Before CKR ™ | | Post CKR ™ | | Post Crosslinking | |
|---|---|---|---|---|---|---|
| Power | 45.07 | D | 42.90 | D | 43.77 | D |
| Shape Factor | 0.651 | | 1.424 | | 0.957 | |
| Radius | 7.49 | mm | 7.87 | mm | 7.71 | mm |
| KD | 0.75 | D | 0.59 | D | 0.82 | D |

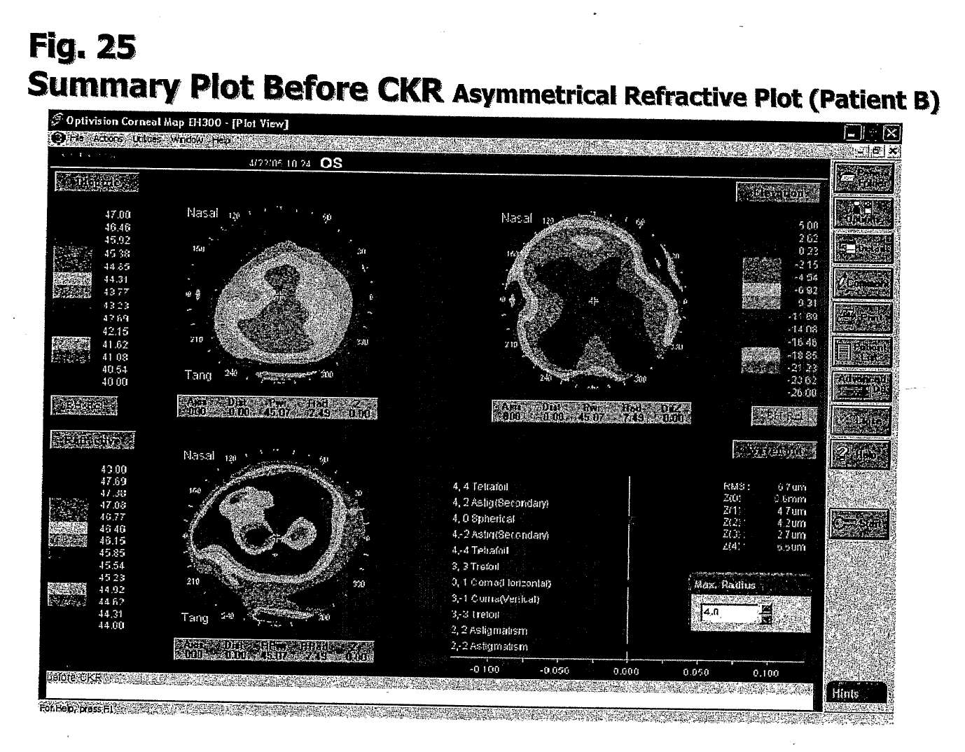

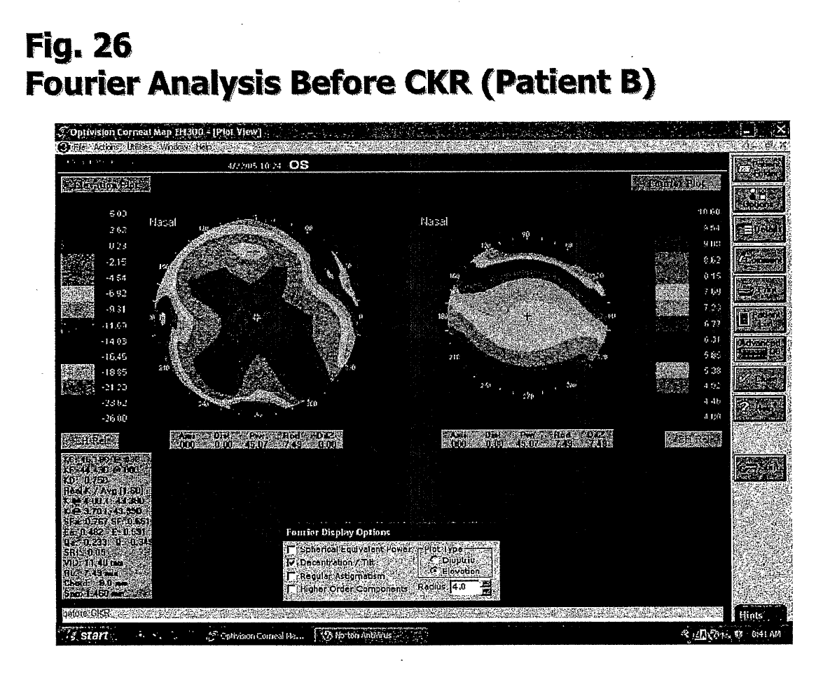

For this patient, the Shape Factor increased after CKR™, but was reduced after the crosslinking procedure. A shown in FIGS. 25-28, the effect of the crosslinking procedure was to improve corneal symmetry in this patient. Viewing a summary plot of an Asymmetrical Refractive analysis of this cornea prior to CKR™, asymmetry is demonstrated, particularly when comparing the Wavefront data at the bottom right side of FIG. 25 showing a coma (vertical) surface aberration. Fourier analysis of the pre-CKR™ data (FIG. 26) indicates decentration or Tilt is present. In contrast, viewing the same plots twelve days after the crosslinking procedure, a more symmetrical refractive plot is shown. While an astigmatism and trefoil aberrations are present, the aberration is flipped from superior to inferior, and centration and symmetry are improved (Compare FIGS. 25 and 27 and FIGS. 26 and 28)

### Representative Patient C

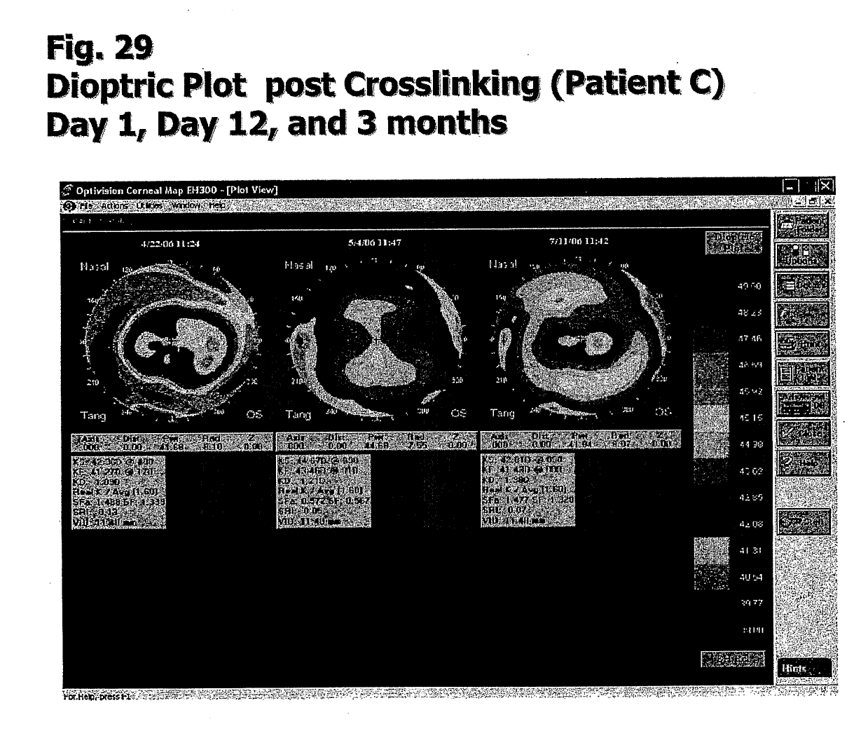

Changes in corneal topography after crosslinking are shown for patient C in FIGS. 29-31. Side by side Dioptric plots of the cornea are shown for day 1 prior to the crosslinking procedure and for post-crosslinking at day 10 and at three months.

The data indicate an improvement in topography with crosslinking. See, for example, the improved KD, from 1.09 D at day 1 to 1.21 D at day 10 and 1.38 D at 3 months post crosslinking (FIG. 29). Refractive error changes from day one to day 10 from 41.68 D to 44.68 D, but returns to pre-crosslinking levels (41.84 D) by three months post-crosslinking, as does the radius of curvature (Rad), 8.10 mm at day 1, 7.55 mm at day 10, and 8.07 mm at three months.

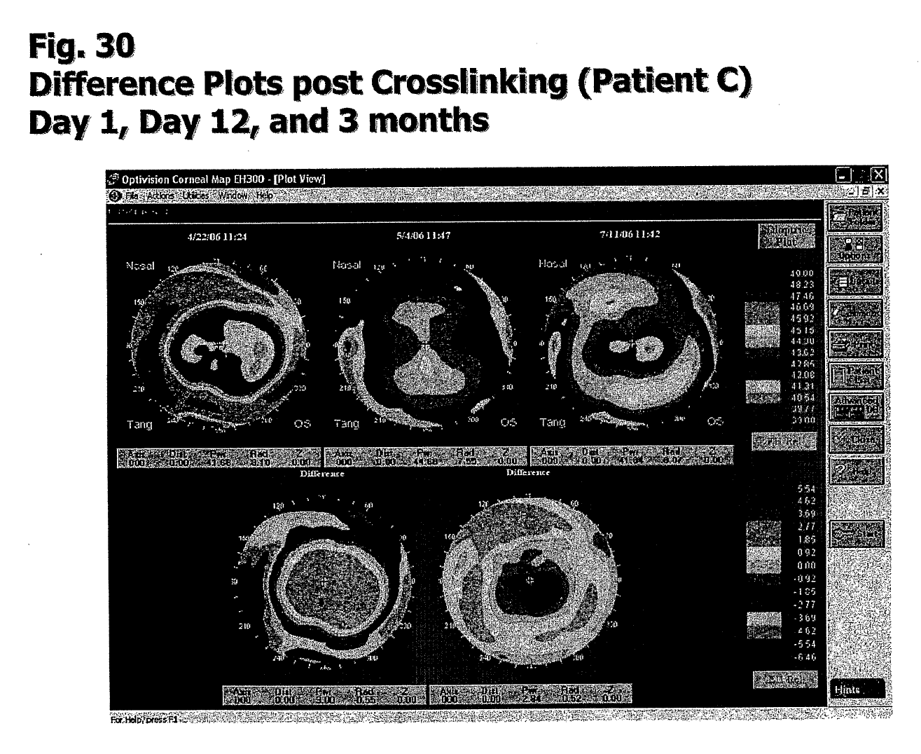

The difference plot shown in FIG. 30 demonstrates an overall improvement in the irregular topography of patient C's cornea. The central falttening zone (blue area) is more centrally located and more symmetrical.

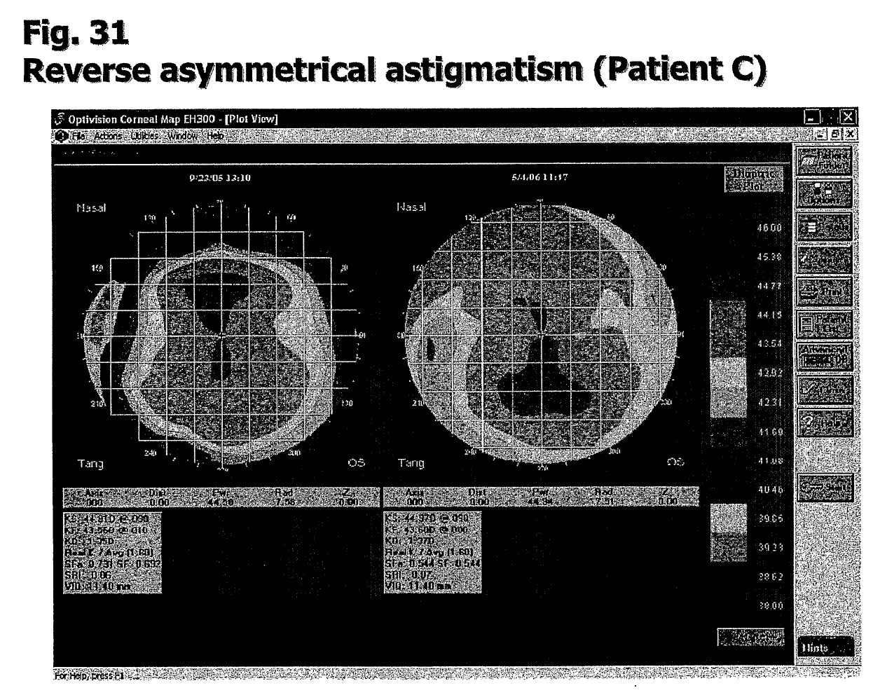

An interesting effect is shown when the corneal topography prior to CKR™ treatment is compared with the 10 day post crosslinking topography. As shown in FIG. 31, a reversal of the patient's asymmetrical astigmatism is demonstrated post crosslinking.

I claim:

1. A method for treating a visual defect of the eye, comprising:
a. providing a corrective contact lens specifically designed for controlled kerato-reformation by computerized corneal topography data obtained from analysis of a patient's cornea;
b. monitoring reformation of the patient's cornea in response to wearing the lens to confirm a desired reformation of the cornea and/or improvement in the patient's vision; and
c. applying a cross-linking agent to the reformed cornea in an amount effective to increase the rigidity of the cornea,
wherein said cross-linking results in maintenance of a desired reformed shape and/or improvement in vision during periods of non-wear of the CKR™ lens.

2. The method of claim 1, further comprising debriding or cutting the cornea's epithelium prior to applying the cross-linking agent.

3. The method of claim 1, wherein said crosslinking agent is riboflavin and UV light.

4. The method of claim 1, wherein said crosslinking agent is riboflavin and UV light at about 360-370 nm.

5. The method of claim 1, wherein said crosslinking agent is riboflavin and UV light at about 365 nm.

6. The method of claim 1, wherein said crosslinking agent is riboflavin and UV light has an intensity of about 3 mW/cm².

7. The method of claim 1, wherein a sufficient amount of the cross-linking agent is applied to the eye to result in the presence of the cross-linking agent in the anterior chamber of the eye.

8. The method of claim 1, wherein a sufficient amount of the cross-linking agent is applied to the eye to result in penetration of the cross-linking agent to at least 300 mm of the cornea.

9. The method of claim 1, further comprising analyzing the eye to confirm the presence of applied cross-linking agent in the anterior chamber of the eye.

10. The method of claim 1, further comprising analyzing the eye via a slit lamp to confirm a yellow color produced by the presence of applied riboflavin in the anterior chamber of the eye.

11. The method of claim 1, further comprising analyzing the eye via a slit lamp to confirm a yellow color produced by the presence of applied riboflavin in the anterior chamber of the eye and in the corneal tissue.

12. The method of claim 1, further comprising removing the corrective contact lens prior to applying the cross-linking agent.

13. The method of claim 1, wherein said desired shape and/or improvement in vision is maintained for at least 6 months.

14. The method of claim 1, wherein said desired shape and/or improvement in vision is maintained for at least 1 year.

15. The method of claim 1, wherein the patient's corneal topography is analyzed using an EH-300 corneal topography map analyzer.

16. The method of claim 1, wherein the method is for correcting one or more of myopia, hyperopia, and astigmatism.

17. The method of claim 1, wherein said applying comprises debriding or cutting the epithelial layer of the cornea to enhance penetration of the crosslinking agent.

18. The method of claim 1, wherein said applying comprises cutting the epithelial layer of the cornea with a scalpel in a pattern that comprises at least two vertical slits and at least one horizontal slit, to enhance penetration of the crosslinking agent.

19. The method of claim 1, wherein said applying comprises cutting the epithelial layer of the cornea with a scalpel in a pattern that comprises at least three vertical slits and at least one horizontal slit, to enhance penetration of the crosslinking agent.

20. The method of claim 1, wherein said cross-linking reduces the periodicity of CKR™ lens wear needed to maintain daily corrected vision.

21. The method of claim 1, wherein said crosslinking reduces the periodicity of CKR™ lens wear needed to maintain daily corrected vision from daily wear to about once per week or to about once per month.
